(19) United States
(12) Patent Application Publication
(10) Pub. No.: US 2016/0194354 A1
(43) Pub. Date: Jul. 7, 2016

(54) STEREOSELECTIVE SYNTHESIS OF DIOLS AND TRIOLS BY MANNICH REACTION AND THEIR USE IN THE SYNTHESIS OF CARFILZOMIB

(71) Applicant: SANDOZ AG, Basel (CH)

(72) Inventors: Kathrin HÖFERL-PRANTZ, Kundl (AT); Thorsten WILHELM, Langkampfen (AT)

(21) Appl. No.: 14/916,521

(22) PCT Filed: Aug. 20, 2014
(86) PCT No.: PCT/EP2014/067728

§ 371 Date: Mar. 3, 2016

(30) Foreign Application Priority Data
Sep. 6, 2013 (EP) 13183306.3

(51) Int. Cl. C07K 5/103; C07D 303/36; C07C 219/06; C07D 319/08; C07C 215/16

(52) U.S. Cl. CPC C07K 5/1008; C07D 319/08; C07C 215/16; C07C 219/06; C07D 303/36

(57) ABSTRACT

It is provided an improved process for preparing Carfilzomib, including novel compounds that can be used as intermediates in the process for preparing Carfilzomib.

Carfilzomib is a tetrapeptide epoxy ketone and a selective proteasome inhibitor. It is an analog of epoxomicin.

The US FDA approved it for relapsed and refractory multiple myeloma. It is marketed under the trade name Kyprolis®.

The chemical name of Carfilzomib is (S)-4-Methyl-N—((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)pentanamide, represented by the following chemical structure:

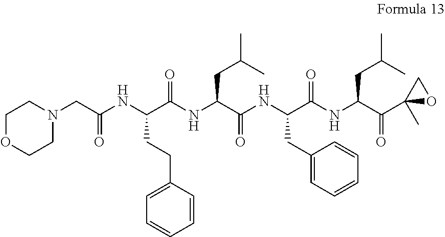 [SMILES: CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)[C@@]1(C)CO1]

A specific route to Carfilzomib is described in WO2005105827 A2 and WO2006017842 A1. Both applications describe as a last step in the synthesis route the coupling of an epoxide of Formula

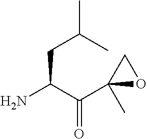

to a peptide of Formula

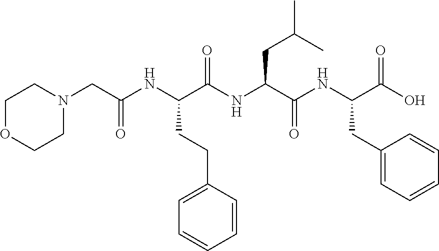 [SMILES: CC(C)C[C@H](NC(=O)[C@H](CCc1ccccc1)NC(=O)CN1CCOCC1)C(=O)N[C@@H](Cc1ccccc1)C(=O)O]

to obtain Carfilzomib. This way the stereocentre of the epoxide is formed in a small molecule. The epoxide is synthetised according to Crews, C. M. et al, Bioorg. Med. Chem. Letter 1999, 9, 2283-2288:

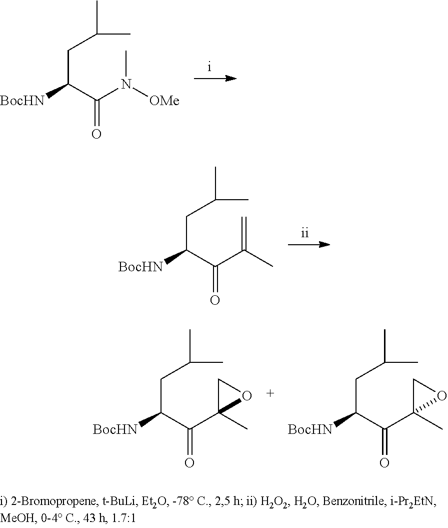

The Boc-protected vinyl ketone is epoxidized in one step with alkaline hydrogen peroxide, leading to a mixture of the diastereomers in a ratio of 1.7:1. The separated diastereomers were obtained after column chromatography.

WO2009045497 describes the same synthesis route to Carfilzomib as WO2005105827 A2 and WO2006017842. Differences are observed in the synthesis of the epoxide building block starting from the vinyl ketone. One route leads from the vinyl ketone over reduction, epoxidation and oxidation to the desired epoxide. This route is also disclosed in WO2005111009. A second route is a one step reaction from the vinyl ketone to the epoxide by an aqueous solution of NaOCl, leading however to a diasteromeric mixture which is purified by column chromatography.

All these synthesis routes leading to Carfilzomib have the disadvantage that the epoxide is formed during the synthesis route as a building block and that the epoxide is not formed with high stereoselectivity, i.e. diastereoselectivity. Hence, the yield of the epoxide building block with the desired configuration is very low. Further, the toxic epoxide building block is formed as an intermediate, which has to be handled over additional steps to obtain the final product.

Hence, it was an object of the present invention to overcome the above-mentioned disadvantages.

It was an object of the present invention to provide a process for preparing Carfilzomib with a high yield and/or a high grade of purity.

Further, the use of hazardous, expensive and dangerous substances should be avoided as much as possible.

Finally, it was an object of the invention to provide substances and/or a process assuring a straightforward reaction and preventing the formation of side products.

### SUMMARY OF THE INVENTION

It was found that the substances and/or method of the present invention could be used to improve the purity, the stereoselectivity and the yield of process, such as the preparation of Carfilzomib. Further advantages of the process of the invention are simple reaction conditions, the use of readily available starting materials and reagents, the use of solvents that are easy to handle and/or easily removed, the prevention of the use of hazardous and explosive materials.

Thus, the above objectives are solved by the provision of an improved process for preparing Carfilzomib, including novel compounds that can be used as intermediates in the process for preparing Carfilzomib, and the provision of processes for preparing intermediates that can be used in the process for preparing Carfilzomib.

One aspect of the invention is a method for producing the epoxide according to Formula 12,

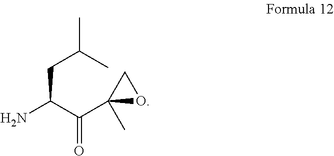

Another aspect of the invention is a method for producing Carfilzomib according to Formula 13,

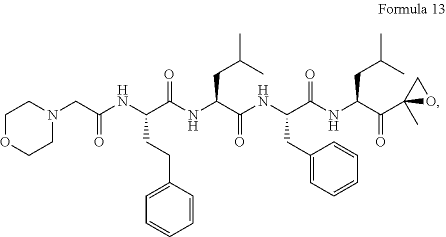 [SMILES: CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)[C@@]1(C)CO1]

wherein the method comprises the compound of Formula 12 as a reactant.

A further aspect of the invention is a method of producing Carfilzomib according to Formula 13 from a compound of Formula 9,

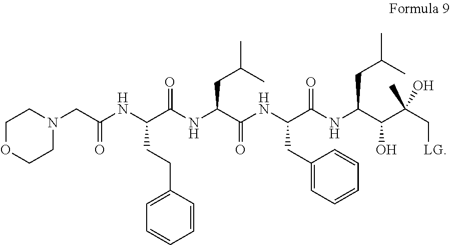

Finally, the invention is directed to a compound selected from the compounds according to any one of Formulae 4, 5, 6, 7, 9, 10 and 11.

### DETAILED DESCRIPTION OF THE INVENTION

Through the provision of the amino function the compound of Formula 12 can be coupled to the carboxy function of the peptide of Formula 8,

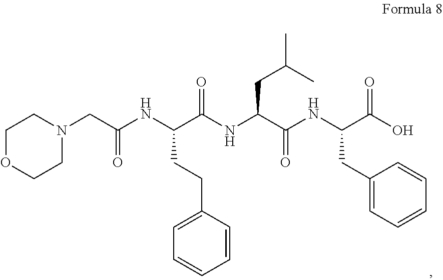 [SMILES: CC(C)C[C@H](NC(=O)[C@H](CCc1ccccc1)NC(=O)CN1CCOCC1)C(=O)N[C@@H](Cc1ccccc1)C(=O)O]

to obtain Carfilzomib according to Formula 13.

It has been found that the epoxide of Formula 12 can be formed with high diastereoselectivity by the method comprising the steps:

a) organocatalytic Mannich-reaction of compounds of Formulae 1, 2 and 3,

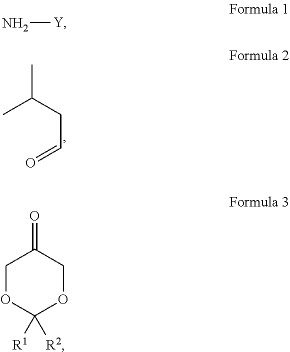

wherein
Y stands for an aromate, heteroaromate or a substituted aromate/heteroaromate,
R¹, R² stand for a C₁-C₉-alkyl, wherein R¹, R² can be connected, forming a ring of 4 to 10 atoms,
leading to a compound of Formula 4,

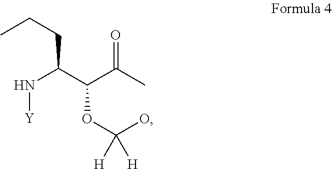

b) methyl addition to the compound of Formula 4, optionally followed by protection of the nitrogen, leading to a compound of Formula 5,

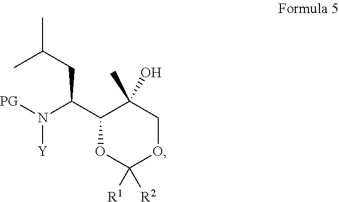

c) deprotection of the compound of Formula 5 to a compound of Formula 6,

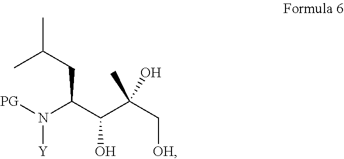

d) transforming the primary alcohol in the compound of Formula 6 into a leaving group leading to a compound of Formula 10,

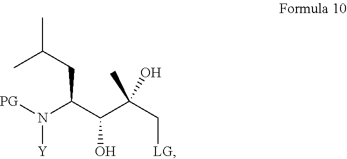

wherein
LG stands for a leaving group,
e) oxidizing the secondary alcohol in the compound of Formula 10 to obtain a compound of Formula 11,

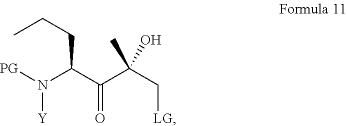

f) epoxide formation by base addition, and
g) deprotection of the amine.

The organocatalytic Mannich reaction of compounds of Formula 1, 2 and 3 can be carried out in an organic solvent or a mixture of an organic solvent with water or in an ionic liquid like bmim.BF4 (1-butyl-3-methylimidazolium tetrafluoroborate). As organic solvent, dimethylsulfoxide (DMSO), dimethylformamide (DMF), toluene, dichloromethane (DCM), N-methyl-2-pyrrolidone (NMP), tetrahydrofurane (THF) or acetonitrile can be used. In an embodiment of the invention, the organic solvent is DMSO.

The compound of Formula 1 is an amino compound having one aromatic moiety. The aromatic moiety can be substituted and/or heteroaromatic. The nitrogen must be however directly connected to the aromatic/heteroaromatic moiety. The aromatic/heteroaromatic moiety can be cleaved off the nitrogen in a later stage of the method according to the invention. Preferably, the aromatic group is p-methoxyphenyl (PMP).

The compound of Formula 3 is an O,O-acetale and is derived from 1,3-dihydroxypropan-2-one. R¹ and R² stand for a C₁-C₉-alkyl, wherein R¹ and R² can be connected to form a ring of 4 to 10 atoms. In one embodiment, the ring has 5 atoms. In a second embodiment, the ring has 6 atoms. The hydrogen atoms of the alkyl may be substituted by any kind of atoms or groups, e.g. halogens, hydroxy functions or nitro functions. One or more carbon atoms of the ring may be substituted by hetero atoms, such as N or O. In a preferred embodiment, the compound of Formula 3 is 2,2-Dimethyl-1,3-dioxan-5-one or 1,5-Dioxaspiro[5.5]undecan-3-one.

The organocatalytic Mannich reaction is carried out with an organocatalyst. In one embodiment, the organo catalyst is an amino acid, such as (L)-alanine or derivatives thereof, (L)-proline or derivatives thereof, such as (L)-prolinol, a trimethylsilyl protected (L)-prolinol or pyrrolidinyltetrazol. Preferably, the organo catalyst is (L)-alanine.

The organocatalytic Mannich reaction provides a Mannich product with high enantio and diastereoselectivity that can be up to >99% ee and de after recrystallization, if necessary.

The methyl addition to the compound of Formula 4 is carried out with nucleophilic methyl compounds. In one embodiment of the invention, the nucleophilic methyl compound is methyl lithium or a Grignard reagent, e.g. methyl magnesium bromide. In a preferred embodiment, the reaction is carried out with methyl magnesium halide, preferably methyl magnesium bromide. Further, the reaction is carried out in a solvent, preferably an organic solvent or a mixture of organic solvents. In one embodiment of the reaction, the organic solvent is an ether, preferably diethyl ether or THF.

In a subsequent optional step, a nitrogen protecting group is introduced. As protecting group (PG), known amino function protecting groups are suitable, preferably amino function protecting groups that are stable to weak acidic conditions (pH 3-5). Examples of a protecting group are carboxybenzyl (Cbz), phthlaloyl (Phth), tetrachlorophthaloyl (TCP), dithiasuccinyl (Dts), Trifluoroacetyl, methoxycarbonyl, ethoxycarbonyl, allyloxycarbonyl (Alloc), 9-fluorenylmethoxycarbonyl (Fmoc), 2-(trimethylsilyl)ethoxycarbonyl (Teoc), 2,2,2-trichloroethoxycarbonyl (Troc), phenylsulfonyl, p-tolylsulfonyl (Ts), 2- and 4-nitrophenylulfonyl (Ns), 2-(trimethylsilyl)ethylsulfonyl (SES), benzoyl (Bz), benzyl (Bn), diphenylmethyl (Dpm), p-methoxybenzyl (PMB), 3,4-dimethoxy benzyl (DMPM), p-methoxyphenyl (PMP) and allyl. Preferably, the protecting group is Bn or Bz. In an embodiment, the protecting group can be cleaved under acidic or basic conditions. In a second embodiment, the protecting group can be cleaved under reductive conditions.

The desired methyl addition product of Formula 5 could be provided with high diastereoselectivity up to >99%. At this stage, all the relevant steric centres of the epoxide of Formula 12 are formed.

The deprotection of the acetal of Formula 5 to the triol of Formula 6 in step c) is carried out under acidic conditions. In one embodiment of the invention, deprotection is carried out in an aqueous solution of an acid or in an aqueous solution of an acid and an organic solvent. In one embodiment, deprotection can be carried out in an aqueous solution of HCl and dimethylformamide (DMF) or methanol (MeOH).

The primary alcohol in Formula 6 is then transformed into a leaving group in step d). This reaction comprises the activation of the primary alcohol by deprotonation to obtain a nucleophilic alcoholate and addition of an electrophile as reactant. The primary alcohol can be transformed into a leaving group LG that is typically used in organic synthesis methods, such as acetate, mesylate, tosylate, pivaloyl group, i-propyl carbonate, halogenide. In a preferred embodiment, the leaving group is mesylate, i-propyl carbonate or acetate. The reaction can be carried out in an organic solvent, such as toluene, DCM and diethyl ether. The deprotonation of the primary alcohol can be carried out with a base, preferably an organic base such as an amine, more preferably triethylamine or diisopropylethylamine (DIPEA).

Further, the step of oxidizing the secondary alcohol in the compound of Formula 10 to obtain a ketone of Formula 11 in step e) can be carried out in an organic solvent, such as DCM, acetonitrile or DMSO. In organic synthesis, a variety of oxidizing reactions of secondary alcohols and oxidizing reagents, respectively, are known that can all be applied in the present invention, such as Swern oxidation, Pfitzner-Moffatt oxidation, Dess-Martin oxidation, Ley oxidation, oxidation using TEMPO and a cooxidant or a hypervalent iodide reagent like 2-Iodoxybenzoic acid (IBX) or Dess-Martin periodinane. In a preferred embodiment, the oxidation reaction is a Dess-Martin oxidation.

Step f) is the formation of an epoxide via the addition of a base, preferably an organic base, such as pyridine, triethylamine or potassium tert-butyrate. Triethylamine is preferably used in combination with mesylate as leaving group. The reaction can be carried out in an organic solvent, such as DCM. The reaction occurs under complete retention of the configuration.

Step g) is the deprotection of the amine function leading to the epoxide of Formula 12. If a nitrogen protecting group has not been introduced after the methyl addition step, only the Y group is cleaved off. The reaction can be carried out in an organic solvent or in a mixture of an organic solvent and water. Suitable organic solvents are for example alcohol and ethers, e.g. methanol, ethanol, propanol, THF and dioxan. In one embodiment of the reaction, the deprotection can be carried out under acidic, basic, or oxidizing conditions. In a second embodiment of the invention, the deprotection can be carried out in the presence of a catalyst, such as Pd/C and/or hydrogen. For Bn as protecting group, the deprotection is preferably carried out under reducing conditions, for example with hydrogen in the presence of Pd/C in water, alcohol or a mixture of both as solvent. For Bz as protecting group, the deprotection can be carried out under acidic, basic, or reducing conditions. Also the group Y can be cleaved off under different conditions. For PMP as Y, the cleaving is preferably carried out with oxidizing reagents, such as cerium ammonium nitrate (CAN), Dess-Martin periodinane and trichloroisocyanuric acid (TCCP), in solvents such as methanol, acetonitril, water or mixtures thereof.

According to a further aspect of the invention, Carfilzomib according to Formula 13 is formed by a method comprising the steps

a) organocatalytic Mannich-reaction of compounds of Formula 1, 2 and 3,

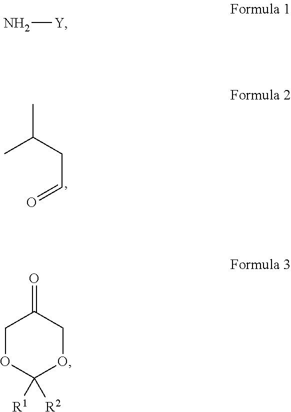

wherein
PG stands for a protecting group,
Y stands for an aromate, heteroaromate or a substituted aromate/heteroaromate,
R¹, R² stand for a C₁-C₉-alkyl, wherein R¹, R² can be connected, forming a ring of 4 to 10 atoms,
leading to a compound of Formula 4,

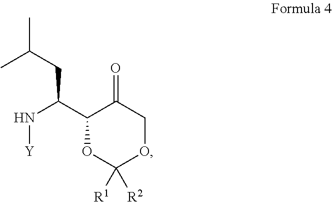

b) methyl addition to the compound of Formula 4, optionally followed by protection of the nitrogen, leading to a compound of Formula 5,

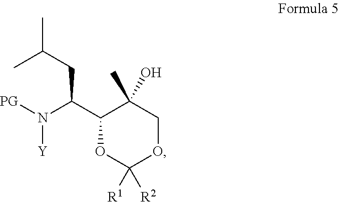

c) deprotection of the compound of Formula 5 to a compound of Formula 6,

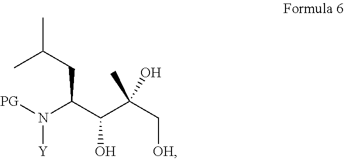

and converting the compound of Formula 6 into Carfilzomib.

The organocatalytic Mannich reaction of compounds of Formula 1, 2 and 3 can be preferably carried out in a solvent, preferably an organic solvent or a mixture of an organic solvent with water or in an ionic liquid like bmim.BF4 (1-butyl-3-methylimidazolium tetrafluoroborate). As organic solvent, dimethylsulfoxide (DMSO), toluene, dichloromethane (DCM), dimethylformamide (DMF), N-methyl-2-pyrrolidone (NMP), tetrahydrofurane (THF) or acetonitrile can be used. In an embodiment of the invention, the organic solvent is DMSO.

The compound of Formula 1 is an amino compound having one aromatic moiety. The aromatic moiety can be substituted and/or heteroaromatic. The nitrogen must be however directly connected to the aromatic/heteroaromatic moiety. The aromatic/heteroaromatic moiety can be cleaved off the nitrogen in a later stage of the method according to the invention. Preferably, the aromatic group is p-methoxyphenyl (PMP).

The compound of Formula 3 is an O,O-acetale and is derived from 1,3-dihydroxypropan-2-one. R¹ and R² stand for an alkyl, wherein R¹ and R² can be connected to form a ring of 4 to 10 atoms. In one embodiment, the ring has 5 atoms. In a second embodiment, the ring has 6 atoms. The hydrogen atoms of the alkyl may be substituted by any kind of atoms or groups, e.g. halogens, hydroxy functions or nitro functions. One or more carbon atoms of the ring may be substituted by hetero atoms, such as N or O. In one embodiment of the invention, the compound of Formula 3 is 2,2-Dimethyl-1,3-dioxan-5-one. In a further embodiment, the compound of Formula 3 is 1,5-Dioxaspiro[5.5]undecan-3-one.

The organocatalytic Mannich reaction is carried out with an organocatalyst. In one embodiment, the organo catalyst is an amino acid. In an embodiment of the invention, the amino acid is (L)-alanine or derivatives thereof, (L)-proline or derivatives thereof, such as (L)-prolinol, a trimethylsilyl protected (L)-prolinol or pyrrolidinyltetrazol. Preferably, the organo catalyst is (L)-alanine.

The organocatalytic Mannich reaction provides a Mannich product with high enantio and diastereoselectivity up to >99% ee and de.

The methyl addition to the compound of Formula 4 is carried out with nucleophilic methyl compounds. In one embodiment of the invention, the nucleophilic methyl compound is methyl lithium or a Grignard reagent, e.g. methyl magnesium bromide. In a preferred embodiment, the reaction is carried out with methyl magnesium halide, preferably methyl magnesium bromide. Further, the reaction is carried out in a solvent, preferably an organic solvent or a mixture of organic solvents. In one embodiment of the reaction, the organic solvent is an ether, preferably diethyl ether or THF.

In a subsequent optional step, a nitrogen protecting group is introduced. As protecting group (PG), known amino function protecting groups are suitable, preferably amino function protecting groups that are stable to weak acidic conditions (pH 3-5). Examples of a protecting group are carboxybenzyl (Cbz), phthlaloyl (Phth), tetrachlorophthaloyl (TCP), dithiasuccinyl (Dts), Trifluoroacetyl, methoxycarbonyl, ethoxycarbonyl, allyloxycarbonyl (Alloc), 9-fluorenylmethoxycarbonyl (Fmoc), 2-(trimethylsilyl)ethoxycarbonyl (Teoc), 2,2,2-trichloroethoxycarbonyl (Troc), phenylsulfonyl, p-tolylsulfonyl (Ts), 2- and 4-nitrophenylulfonyl (Ns), 2-(trimethylsilyl)ethylsulfonyl (SES), benzoyl (Bz), benzyl (Bn), diphenylmethyl (Dpm), p-methoxybenzyl (PMB), 3,4-dimethoxy benzyl (DMPM), p-methoxyphenyl (PMP) and allyl. Preferably, the protecting group is Bn or Bz. In an embodiment, the protecting group can be cleaved under acidic or basic conditions. In a second embodiment, the protecting group can be cleaved under reductive conditions.

The desired methyl addition product of Formula 5 can be provided with high diastereoselectivity up to >99% de. At this stage, the relevant steric information at the epoxide bearing end of Carfilzomib is formed.

The deprotection of the acetal of Formula 5 to the triol of Formula 6 is carried out under acidic conditions. In one embodiment of the invention, deprotection is carried out in an aqueous solution of an acid or in an aqueous solution of an acid and an organic solvent. In one embodiment, deprotection can be carried out in an aqueous solution of HCl and dimethylformamide (DMF) or methanol (MeOH).

Starting from the compound of Formula 6, different routes can lead to Carfilzomib.

In one embodiment, the method for producing Carfilzomib comprising the steps a)-c) further comprises the steps of

d1) transforming the primary alcohol in the compound of Formula 6 into a leaving group,
e1) deprotecting the amino-function to obtain a compound of Formula 7

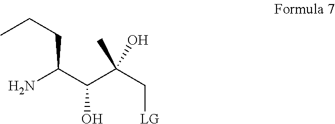

wherein
LG stands for a leaving group
f1) coupling the compound of Formula 7 to the peptide of Formula 8,

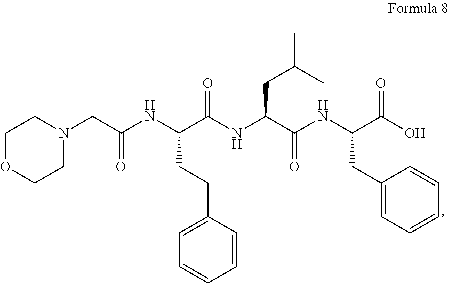

leading to a peptide of Formula 9

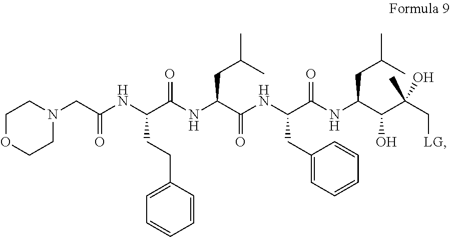

and converting the compound of Formula 9 into Carfilzomib.

The step d1) comprises the activation of the primary alcohol by deprotonation to obtain a nucleophilic alcoholate and addition of an electrophile as reactant. The primary alcohol can be transformed into a leaving group LG that is typically used in organic synthesis methods, such as acetate, mesylate, tosylate, pivaloyl group, i-propyl carbonate, halogenide. In a preferred embodiment, the leaving group is mesylate, i-propyl carbonate or acetate. The reaction can be carried out in an organic solvent, such as toluene and diethyl ether. The deprotonation of the primary alcohol can be carried out with a base, preferably an organic base such as an amine, more preferably triethylamine.

The step e1) is the deprotection of the amine function leading to the compound of Formula 7. If a nitrogen protecting group has not been introduced after the methyl addition step, only the Y group is cleaved off. The reaction can be carried out in an organic solvent or in a mixture of an organic solvent and water. Suitable organic solvents are for example alcohol, ethers, e.g. methanol, ethanol, propanol, THF and dioxan, and dichloromethane. In a preferred embodiment, the deprotection is carried out in THF. In one embodiment of the reaction, the deprotection can be carried out under acidic, basic, or oxidizing conditions, preferably basic conditions. In a second embodiment of the invention, the deprotection can be carried out in the presence of a catalyst, such as Pd/C and/or hydrogen. For Bn as protecting group, the deprotection is preferably carried out under reducing conditions, for example with hydrogen in the presence of Pd/C in water, alcohol or a mixture of both as solvent. For Bz as protecting group, the deprotection can be carried out under acidic, basic, or reducing conditions. Also the group Y can be cleaved off under different conditions. For PMP as Y, the cleaving is preferably carried out with oxidizing reagents, such as cerium ammonium nitrate (CAN), Dess-Martin periodinane and trichloroisocyanuric acid (TCCP), in solvents such as methanol, acetonitril, water or mixtures thereof.

Step f1) is the coupling of compound 7 to the peptide of compound 8. The peptide bond formation can be carried out according to known procedures. In one embodiment, the carboxy function is activated by a coupling agent such as a carbodiimide and/or a triazol. Examples of coupling agents are DCC (dicyclohexylcarbodiimide), DIC (diisopropylcarbodiimide), HOBt (1-hydroxy-benzotriazole), HOAt (1-hydroxy-7-aza-benzotriazole), BOP (benzotriazol-1-yloxy)tris(dimethylamino)phosphonium hexafluorophosphate), PyBOP (benzotriazol-1-yloxy)tris(pyrrolidino)phosphonium hexafluorophosphat, PyBroP (bromo)tris(pyrrolidino)phosphonium hexafluorophosphate), BroP (bromo)tris(dimethylamino)phosphonium hexafluorophosphate), HBTU (2-(1H-benzotriazole-1-yl)-1,1,3,3-tetramethyluronium hexafluorophosphate) and mixtures thereof. In a preferred embodiment, the coupling reagent is DCC and HOBt. Additionally it is preferred that an organic alkaline substance, preferably an amine, is present in the mixture. Examples of the organic alkaline substance are DBU (1,8-diazabicyclo[5.4.0]undec-7-en), triethylamine and DIPEA (diisopropylethylamin), in particular DIPEA. The reaction can be carried out in an organic solvent, such as acetonitrile, DCM and DMF, preferably DCM. In one embodiment, the solvent is a mixture of at least two organic solvents, such as DCM/DMF.

Starting from the peptide of Formula 9, Carfilzomib can be obtained in at least two ways. In one embodiment, the method of steps d1) to f1) further comprises the steps of

g1.1) epoxide formation by base addition, and
h1.1) oxidation of the secondary alcohol.

In an another embodiment, the method of steps d1) to f1) further comprises the steps of

g1.2) oxidation of the secondary alcohol in the compound of Formula 9, and
h1.2) epoxide formation by base addition.

The steps g1.1) and h1.2) are carried out in an organic solvent, such as DCM or an ether such as THF or diethyl ether, preferably DCM. The epoxide is formed upon addition of a base. In one embodiment, the base is an organic base, such as pyridine or NaOtBu/KOtBu. In one embodiment, the epoxide is formed at room temperature. The epoxide formation is formed under retention of the configuration.

Steps h1.1) and g1.2) can be carried out in an organic solvent, such as DCM, acetonitrile or DMSO. In organic synthesis, a variety of oxidizing reactions of secondary alcohols and oxidizing reagents, respectively, are known that can all be applied in the present invention, such as Swern oxidation, Pfitzner-Moffatt oxidation, Dess-Martin oxidation, Ley oxidation, oxidation using TEMPO and cooxidants or hypervalent iodide reagents like 2-Iodoxybenzoic acid (MX), Dess-Martin periodinane (DMP). In a preferred embodiment, the oxidation reaction is a DMP or IBX oxidation.

In a further embodiment of the reaction, the method of forming Carfilzomib of steps a) to c) further comprises the steps of

d2) transforming the primary alcohol in the compound of Formula 6 into a leaving group leading to a compound of Formula 10,

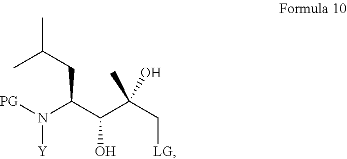

wherein
LG stands for a leaving group
PG stands for a protecting group,
Y stands for an aromate, heteroaromate or a substituted aromate/heteroaromate,
e2) oxidizing the secondary alcohol in the compound of Formula 10 to obtain a compound a Formula 11

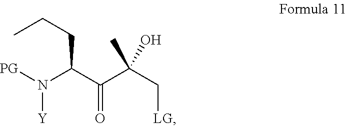

f2) epoxide formation by base addition,
wherein the steps e2) and f2) can be carried out in any order,
g2) deprotection of the amine leading to an epoxide of Formula 12,

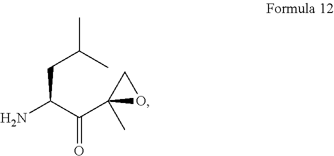

h2) coupling of the epoxide of Formula 12 to a peptide of Formula 8,

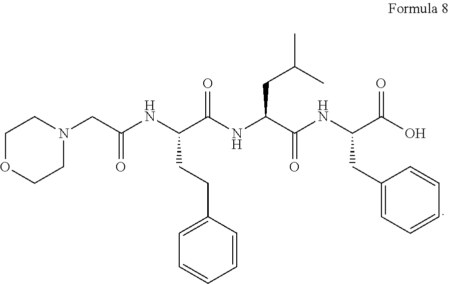

The reaction of steps d2) comprises the activation of the primary alcohol by deprotonation to obtain a nucleophilic alcoholate and addition of an electrophile as reactant. The primary alcohol can be transformed into a leaving group LG that is typically used in organic synthesis methods, such as acetate, mesylate, tosylate, pivaloyl group, i-propyl carbonate, halogenide. In a preferred embodiment, the leaving group is mesylate, i-propyl carbonate or acetate. The reaction can be carried out in an organic solvent, such as toluene and diethyl ether. The deprotonation of the primary alcohol can be carried out with a base, preferably an organic base such as an amine, more preferably triethylamine or DIPEA.

Step e2) can be carried out in an organic solvent, such as DCM, acetonitrile or DMSO. In organic synthesis, a variety of oxidizing reactions of secondary alcohols and oxidizing reagents, respectively, are known that can all be applied in the present invention, such as Swern oxidation, Pfitzner-Moffatt oxidation, Dess-Martin oxidation, Ley oxidation, oxidation using TEMPO and cooxidants or hypervalent iodide reagents like 2-Iodoxybenzoic acid (IBX), Dess-Martin periodinane (DMP). In a preferred embodiment, the oxidation reaction is a DMP or IBX oxidation.

Step f2) can be carried out in an organic solvent, such as DCM or an ether such as THF or diethylether, preferably DCM. The epoxide is formed upon addition of a base. In one embodiment, the base is an organic base, such as pyridine, or sodium/potassium tert-butanolate. In one embodiment, the epoxide is formed at room temperature. The epoxidation is formed under retention of the configuration.

Step g2) can be carried out in an organic solvent or in a mixture of an organic solvent and water. Suitable organic solvents are for example alcohol and ethers, e.g. methanol, ethanol, propanol, THF and dioxan. Preferably, the solvent is THF. In one embodiment of the reaction, the deprotection can be carried out under strong acidic, basic, or oxidizing conditions. In a second embodiment of the invention, the deprotection can be carried out in the presence of a catalyst, such as Pd/C and/or hydrogen. For Bn as protecting group, the deprotection is preferably carried out under reducing conditions, for example with hydrogen in the presence of Pd/C in water, alcohol or a mixture of both as solvent. For Bz as protecting group, the deprotection can be carried out under acidic, basic, or reducing conditions. Also the group Y can be cleaved off under different conditions. For PMP as Y, the cleaving is preferably carried out with oxidizing reagents, such as cerium ammonium nitrate (CAN), Dess-Martin periodinane and trichloroisocyanuric acid (TCCP), in solvents such as methanol, acetonitril, water or mixtures thereof.

Step h2) can be carried out according to known procedures, e.g. according to WO 2005/105827. The peptide bond formation can be carried out in an organic solvent, such as acetonitrile, DCM, DMF, DMSO, DMPU, preferably DMF. In one embodiment, the solvent is a mixture of at least two organic solvents, such as DCM/DMF. In one embodiment, the carboxy function of the peptide of formula 8 is activated by a coupling agent such as a carbodiimide and/or a triazol. Examples of coupling agents are DCC (dicyclohexylcarbodiimide), DIC (diisopropylcarbodiimide), HOBt (1-hydroxy-benzotriazole), HOAt (1-hydroxy-7-aza-benzotriazole), BOP (benzotriazol-1-yloxy)tris(dimethylamino)phosphonium hexafluorophosphate), PyBOP (benzotriazol-1-yloxy)tris(pyrrolidino)phosphonium hexafluorophosphat, PyBroP (bromo)tris(pyrrolidino)phosphonium hexafluorophosphate), BroP (bromo)tris(dimethylamino)phosphonium hexafluorophosphate), HBTU (2-(1H-benzotriazole-1-yl)-1,1,3,3-tetramethyluronium hexafluorophosphate) and mixtures thereof. Additionally, it is preferred that an organic alkaline substance, preferably an amine, is present in the mixture. Examples of the organic alkaline substance are triethylamine and DIPEA (diisopropylethylamin), in particular DIPEA.

A further aspect of the invention is a method for producing Carfilzomib according to Formula 13,

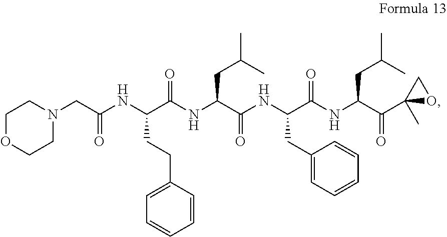 [SMILES: CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)[C@@]1(C)CO1]

from a compound of Formula 9,

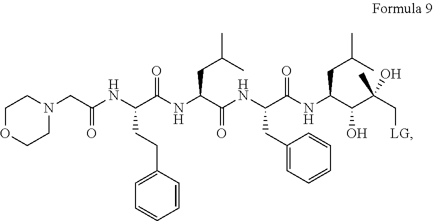

wherein
LG stands for a leaving group,
wherein the method comprises the steps of
i) epoxide formation by base addition, and
ii) oxidation of the secondary alcohol,
wherein the steps i) and ii) can be carried out in any order.

Steps i) and ii) correspond to steps g1.1), h1.1) and g1.2), h1.2) mentioned above, respectively. The compound of Formula 9 can be formed by reaction of a compound of Formula 7,

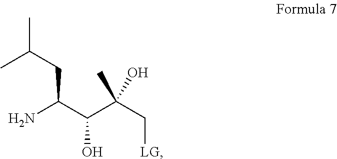

wherein
LG stands for a leaving group,
with a compound of Formula 8,

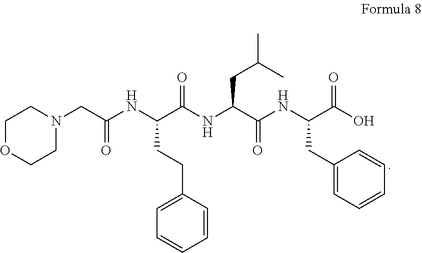 [SMILES: CC(C)C[C@H](NC(=O)[C@H](CCc1ccccc1)NC(=O)CN1CCOCC1)C(=O)N[C@@H](Cc1ccccc1)C(=O)O]

The reaction between the compound of Formula 7 and the compound of Formula 8 is a peptide bond formation that can be carried out according to known procedures. The peptide bond formation can be carried out in an organic solvent, such as acetonitrile, DCM, DMF, DMSO, DMPU, preferably DMF. In one embodiment, the solvent is a mixture of at least two organic solvents, such as DCM/DMF. In one embodiment, the carboxy function of the peptide of formula 8 is activated by a coupling agent such as a carbodiimide and/or a triazol. Examples of coupling agents are DCC (dicyclohexylcarbodiimide), DIC (diisopropylcarbodiimide), HOBt (1-hydroxy-benzotriazole), HOAt (1-hydroxy-7-aza-benzotriazole), BOP (benzotriazol-1-yloxy)tris(dimethylamino)phosphonium hexafluorophosphate), PyBOP (benzotriazol-1-yloxy)tris(pyrrolidino)phosphonium hexafluorophosphat, PyBroP (bromo)tris(pyrrolidino)phosphonium hexafluorophosphate), BroP (bromo)tris(dimethylamino)phosphonium hexafluorophosphate), HBTU (2-(1H-benzotriazole-1-yl)-1,1,3,3-tetramethyluronium hexafluorophosphate) and mixtures thereof. Additionally, it is preferred that an organic alkaline substance, preferably an amine, is present in the mixture. Examples of the organic alkaline substance are triethylamine and DIPEA (diisopropylethylamin), in particular DIPEA.

A further aspect of the invention is a method of producing Carfilzomib according to Formula 13 from a compound of Formula 11,

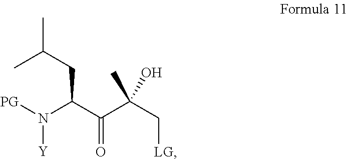

wherein
LG stands for a leaving group
PG stands for a protecting group
Y stands for an aromate, heteroaromate or a substituted aromate/heteroaromate,
comprising the steps:
i) epoxide formation by base addition,
ii) deprotection of the amine leading to an epoxide of Formula 12

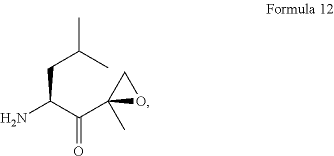

iii) coupling of the epoxide of Formula 12 to a peptide of Formula 8,

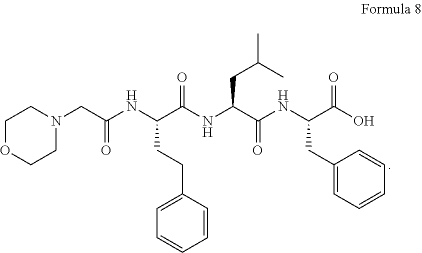 [SMILES: CC(C)C[C@H](NC(=O)[C@H](CCc1ccccc1)NC(=O)CN1CCOCC1)C(=O)N[C@@H](Cc1ccccc1)C(=O)O]

The leaving group LG of the compound of Formula 11 is a LG that is typically used in organic synthesis methods, such as acetate, mesylate, tosylate, pivaloyl group, i-propyl carbonate, halogenide. Preferably, the leaving group is mesylate, i-propyl carbonate or acetate.

As protecting group PG in the compound of Formula 11, known amino function protecting groups are suitable, preferably amino function protecting groups that are stable to acidic conditions. For example, carboxybenzyl (Cbz), phthlaloyl (Phth), tetrachlorophthaloyl (TCP), dithiasuccinyl (Dts), Trifluoroacetyl, methoxycarbonyl, ethoxycarbonyl, allyloxycarbonyl (Alloc), 9-fluorenylmethoxycarbonyl (Fmoc), 2-(trimethylsilyl)ethoxycarbonyl (Teoc), 2,2,2-trichloroethoxycarbonyl (Troc), phenylsulfonyl, p-tolylsulfonyl (Ts), 2- and 4-nitrophenylulfonyl (Ns), 2-(trimethylsilyl)ethylsulfonyl (SES), benzoyl (Bz), benzyl (Bn), diphenylmethyl (Dpm), p-methoxybenzyl (PMB), 3,4-dimethoxy benzyl (DMPM), p-methoxyphenyl (PMP) and allyl can be used as protecting groups. In a preferred embodiment, the protecting group is Bz or Bn. In an embodiment, the protecting group can be cleaved under acidic or basic conditions. In a second embodiment, the protecting group can be cleaved under reductive conditions.

The epoxide formation of step i) can be carried out in an organic solvent, such as DCM or an ether such as THF or diethylether, preferably DCM. The epoxide is formed upon addition of a base. In one embodiment, the base is an organic base, such as pyridine, triethylamine or sodium/potassium tert-butanolate. In one embodiment, the epoxide is formed at room temperature. The epoxide is formed under retention of the configuration.

The step ii) is the deprotection of the amine function leading to the compound of Formula 12. The reaction can be carried out in an organic solvent or in a mixture of an organic solvent and water. Suitable organic solvents are for example alcohol and ethers, e.g. methanol, ethanol, propanol, THF and dioxan. In a preferred embodiment, the solvent is THF. In one embodiment of the reaction, the deprotection can be carried out under acidic, basic, or oxidizing conditions. In a second embodiment of the invention, the deprotection can be carried out in the presence of a catalyst, such as Pd/C and/or hydrogen. For Bn as protecting group, the deprotection is preferably carried out under reducing conditions, for example with hydrogen in the presence of Pd/C in water, alcohol or a mixture of both as solvent. For Bz as protecting group, the deprotection can be carried out under acidic, basic, or reducing conditions. Also the group Y can be cleaved off under different conditions. For PMP as Y, the cleaving is preferably carried out with oxidizing reagents, such as cerium ammonium nitrate (CAN), Dess-Martin periodinane and trichloroisocyanuric acid (TCCP), in solvents such as methanol, acetonitril, water or mixtures thereof.

Step iii) is a peptide bond formation that can be carried out according to known procedures. The peptide bond formation can be carried out in an organic solvent, such as acetonitrile, DCM, DMF, DMSO, DMPU, preferably DMF. In one embodiment, the solvent is a mixture of at least two organic solvents, such as DCM/DMF. In one embodiment, the carboxy function of the peptide of formula 8 is activated by a coupling agent such as a carbodiimide and/or a triazol. Examples of coupling agents are DCC (dicyclohexylcarbodiimide), DIC (diisopropylcarbodiimide), HOBt (1-hydroxy-benzotiazole), HOAt (1-hydroxy-7-aza-benzotiazole), BOP (benzotriazol-1-yloxy)tris(dimethylamino)phosphonium hexafluorophosphate), PyBOP (benzotriazol-1-yloxy)tris(pyrrolidino)phosphonium hexafluorophosphat, PyBroP (bromo)tris(pyrrolidino)phosphonium hexafluorophosphate), BroP (bromo)tris(dimethylamino)phosphonium hexafluorophosphate), HBTU (2-(1H-benzotiazole-1-yl)-1,1,3,3-tetramethyluronium hexafluorophosphate) and mixtures thereof. Additionally, it is preferred that an organic alkaline substance, preferably an amine, is present in the mixture. Examples of the organic alkaline substance are triethylamine and DIPEA (diisopropylethylamin), in particular DIPEA.

Finally, the invention is directed to a compound selected from the compounds according to any one of Formulae 4, 5, 6, 7, 9, 10 and 11. These compounds are intermediates in the novel synthesis of Carfilzomib and enable to obtain Carfilzomib with high stereoselectivity.

Another aspect of the invention is a method for preparing epoxides of formula 20

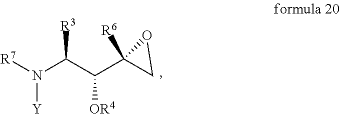

with high stereoselectivity, comprising the steps:
a) organocatalytic Mannich-reaction of compounds of formula 1, 14 and 15,

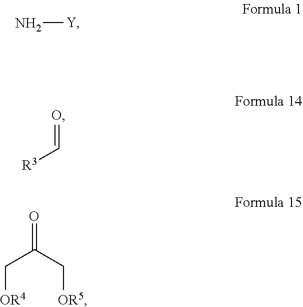

wherein
Y stands for an aryl, heteroaryl or a substituted aryl/heteroaryl,
R³ stands for H, ketone, ester, acetal, unbranched or branched C₁₋₂₀-(hetero)alkyl, C₁₋₂₀-(hetero)alkenyl, C₁₋₂₀-(hetero)alkynyl, (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkenyl, C₃₋₂₀-cyclo(hetero)alkynyl, any of which is optionally further substituted, the heteroatom is selected from O, N and/or S,
R⁴, R⁵ are independently selected from H, unbranched or branched C₁₋₂₀-(hetero)alkyl, C₁₋₂₀-(hetero)alkenyl, C₁₋₂₀-(hetero)alkynyl, (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkenyl, C₃₋₂₀-cyclo(hetero)alkynyl, benzyl, protecting groups for alcohols such as silyl groups, ester, carbonates, sulfates, acetals, wherein R⁴, R⁵ can be connected by one carbon atom, eventually being part of a ring of 4, 5, 6 or 7 atoms,
leading to a compound of formula 16.

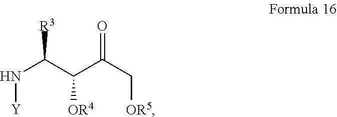

b) stereoselective addition of R⁶ to the compound of Formula 16, optionally followed by protection of the nitrogen, leading to a compound of formula 17,

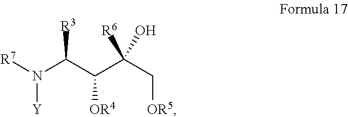

wherein
R⁶, R⁷ are independently selected from H, branched or unbranched C₁₋₂₀-(hetero)alkyl, benzyl, benzoyl, aryl-C₁₋₂₀-(hetero)alkyl, (hetero)aryl,
c) deprotection of the compound of Formula 17 to a compound of Formula 18,

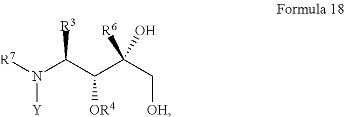

d) transforming the primary alcohol in the compound of Formula 18 into a leaving group, leading to a compound of Formula 19,

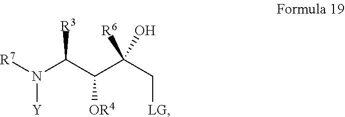

wherein
LG stands for a leaving group such as acetate, halogen substituted acetate, mesylate, tosylate, pivaloyl group, i-propyl carbonate, chloride, bromide, iodide,
and
e) epoxide formation by base addition.

The organocatalytic Mannich reaction of compounds of Formula 1, 14 and 15 can be carried out in an organic solvent or a mixture of an organic solvent with water or in an ionic liquid like bmim.BF4 (1-butyl-3-methylimidazolium tetrafluoroborate). As organic solvent, dimethylsulfoxide (DMSO), dimethylformamide (DMF), toluene, dichloromethane (DCM), N-methyl-2-pyrrolidone (NMP), tetrahydrofurane (THF) or acetonitrile can be used. In an embodiment of the invention, the organic solvent is DMSO.

The compound of Formula 1 is an amino compound having one aromatic moiety. The aromatic moiety can be substituted and/or heteroaromatic. The nitrogen must be however directly connected to the aromatic/heteroaromatic moiety. Optionally, the aromatic/heteroaromatic moiety can be cleaved off the nitrogen in a later stage of the method according to the invention. Preferably, the aromatic group is p-methoxyphenyl (PMP).

The organocatalytic Mannich reaction is carried out with an organocatalyst. In one embodiment, the organo catalyst is an amino acid, such as (L)-alanine or derivatives thereof, (L)-proline or derivatives thereof, such as (L)-prolinol, a trimethylsilyl protected (L)-prolinol or pyrrolidinyltetrazol. Preferably, the organo catalyst is (L)-alanine.

The organocatalytic Mannich reaction provides a Mannich product of Formula 16 with high enantio- and diastereoselectivity up to >99% ee and de.

The addition of R⁶ to the compound of Formula 16 is carried out with nucleophilic compounds and is stereoselective. In one embodiment of the invention, the nucleophilic compound is a nucleophilic methyl compound. In one embodiment of the invention, the nucleophilic methyl compound is methyl lithium or a Grignard reagent, e.g. methyl magnesium bromide. In a preferred embodiment, the reaction is carried out with methyl magnesium halide, preferably methyl magnesium bromide. Further, the reaction is carried out in a solvent, preferably an organic solvent or a mixture of organic solvents. In one embodiment of the reaction, the organic solvent is an ether, preferably diethyl ether or THF.

In a subsequent optional step, a group R⁷ is introduced. R⁷ is selected from H, branched or unbranched C₁₋₂₀-(hetero)alkyl, benzyl (Bn), benzoyl (Bz), aryl-C₁₋₂₀-(hetero)alkyl, (hetero)aryl. Preferably, R⁷ is Bn or Bz. In an embodiment, R⁷ can be cleaved under acidic or basic conditions. In a second embodiment, R⁷ can be cleaved under reductive conditions.

The desired addition product of Formula 17 can be provided with high diastereoselectivity up to >99%. At this stage, all the relevant steric centres of the epoxide of Formula 20 are formed.

The primary alcohol in Formula 18 is transformed into a leaving group in step d). This reaction comprises the activation of the primary alcohol by deprotonation to obtain a nucleophilic alcoholate and addition of an electrophile as reactant. The primary alcohol can be transformed into a leaving group LG that is typically used in organic synthesis methods, such as acetate, mesylate, tosylate, pivaloyl group, i-propyl carbonate, halogenide. In a preferred embodiment, the leaving group is mesylate, i-propyl carbonate or acetate. The reaction can be carried out in an organic solvent, such as toluene, DCM and diethyl ether. The deprotonation of the primary alcohol can be carried out with a base, preferably an organic base such as an amine, more preferably triethylamine or diisopropylethylamine (DIPEA).

Step e) is the formation of an epoxide via the addition of a base, preferably an organic base, such as pyridine, triethylamine or potassium tert-butyrate. Triethylamine is preferably used in combination with mesylate as leaving group. The reaction can be carried out in an organic solvent, such as DCM. The reaction occurs under complete retention of the configuration.

A further aspect is a method of preparing a compound of Formula 18,

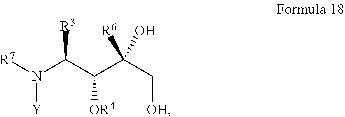

wherein
Y stands for an aryl, heteroaryl or a substituted aryl/heteroaryl,
R³ stands for H, ketone, ester, acetal, unbranched or branched C₁₋₂₀-(hetero)alkyl, C₁₋₂₀-(hetero)alkenyl, C₁₋₂₀-(hetero)alkynyl, (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkenyl, C₃₋₂₀-cyclo(hetero)alkynyl, any of which is optionally further substituted, the heteroatom is selected from O, N and/or S,
R⁴ is selected from H, unbranched or branched C₁₋₂₀-(hetero)alkyl, C₁₋₂₀-(hetero)alkenyl, C₁₋₂₀-(hetero)alkynyl, (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkenyl, C₃₋₂₀-cyclo(hetero)alkynyl, benzyl, protecting groups for alcohols such as silyl groups, ester, carbonates, sulfates, acetals,
R⁶ is selected from branched or unbranched C₁₋₂₀-(hetero)alkyl, benzyl, benzoyl, aryl-C₁₋₂₀-(hetero)alkyl, (hetero)aryl
R⁷ is selected from H, branched or unbranched C₁₋₂₀-(hetero)alkyl, benzyl, benzoyl, aryl-C₁₋₂₀-(hetero)alkyl, (hetero)aryl,
using a compound of Formula 16

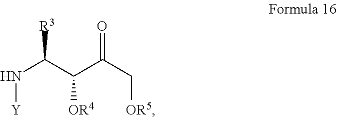

wherein
R⁵ is selected from H, unbranched or branched C₁₋₂₀-(hetero)alkyl, C₁₋₂₀-(hetero)alkenyl, C₁₋₂₀-(hetero)alkenyl, (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkenyl, C₃₋₂₀-cyclo(hetero)alkynyl, benzyl, protecting groups for alcohols such as silyl groups, ester, carbonates, sulfates, acetals, wherein R⁴, R⁵ can be connected by one carbon atom, eventually being part of a ring of 4, 5, 6 or 7 atoms.

Yet further, an aspect of the present invention is a method for preparing a compound of Formula 16,

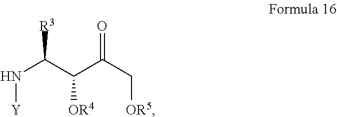

comprising an an organocatalytic Mannich-reaction of compounds of formula 1, 14 and 15,

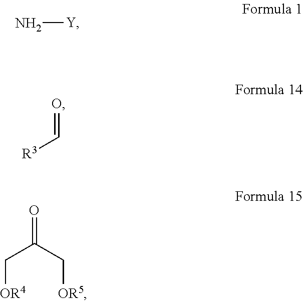

wherein
Y stands for an awl, heteroaryl or a substituted aryl/heteroaryl,
R³ stands for ketone, unbranched C₁₋₂₀-(hetero)alkyl, C₁₋₂₀-(hetero)alkenyl, C₁₋₂₀-(hetero)alkynyl, heteroaryl-C₁₋₂₀-(hetero)alkyl, C₃₋₂₀-cyclo(hetero)alkyl, C₃₋₂₀-cyclo(hetero)alkenyl, C₃₋₂₀-cyclo(hetero)alkynyl, any of which is optionally further substituted, the heteroatom is selected from O, N and/or S,
R⁴, R⁵ are independently selected from H, unbranched or branched C₁₋₂₀-(hetero)alkyl, C₁₋₂₀-(hetero)alkenyl, C₁₋₂₀-(hetero)alkynyl, (hetero)aryl, aryl-C₁₋₂₀-(hetero)alkyl, heteroaryl-C₁₋₂₀-(hetero)alkyl, C₃₋₂₀-cyclo(hetero)alkyl, C₃₋₂₀-cyclo(hetero)alkenyl, C₃₋₂₀-cyclo(hetero)alkynyl, benzyl, protecting groups for alcohols such as silyl groups, ester, carbonates, sulfates, acetals, wherein R⁴, R⁵ can be connected by one carbon atom, eventually being part of a ring of 4, 5, 6 or 7 atoms.

In the methods for preparing a compound of Formula 20, 18 or 16, respectively, R³ is preferably a ketone, methyl, ethyl, n-propyl, n-butyl, isobutyl, t-butyl, neo-pentyl, sec-pentyl, 3-pentyl, t-pentyl, isopentyl, n-pentyl, a linear or branched C₆₋₂₀-(hetero)alkyl, C₁₋₂₀-(hetero)alkenyl, C₁₋₂₀-(hetero)alkynyl, heteroaryl-C₁₋₂₀-(hetero)alkyl, C₃₋₂₀-cyclo(hetero)alkyl, C₃₋₂₀-cyclo(hetero)alkenyl, C₃₋₂₀-cyclo(hetero)alkynyl, any of which is optionally further substituted, the heteroatom is selected from O, N and/or S.

As the organocatalytic Mannich reaction leading to the compounds of Formula 4 and 16 with excellent diastereo- and enantioselectivity, these compounds can be obtained with a ee and de of >99%, eventually after a recrystallization step, if necessary.

### EXAMPLES

### Example 1

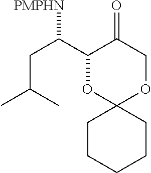

p-Anisidine (21.17 g, 171.9 mmol) was dissolved in dmso (650 mL) and isovaleraldehyde (13.46 g, 156.27 mmol) was added and stirred for 10 min. Then (L)-alanine (13.92 g, 156.27 mmol) and water (5.63 g, 312.54 mmol) was added and stirred for 10 min followed by the addition of the ketone (53.2 g, 312.54 mmol). After 7 days alanine was removed from the heterogenous mixture by filtration. Water and EtOAc was added and the mixture was extracted with EtOAc. The combined organic layer was dried over Na₂SO₄ and the solvent removed under reduced pressure. Purification by crystallization from EtOH, followed by washing with heptane gave 28.4 g (51%) the pure product (>99% ee).

¹H NMR (500 MHz, d⁶-dmso) δ=6.68 (d, J=9.10 Hz, 2H), 6.61 (d, J=9.15 Hz, 2H), 4.51 (d, J=10.40 Hz, 1H), 4.35 (s, 1H), 4.19 (dd, J=1.45, 16.55 Hz, 1H), 3.93 (d, J=16.70 Hz, 1H), 3.82 (m, 1H), 3.62 (s, 3H), 1.86 (m, 1H), 1.77 (m, 1H), 1.69-1.54 (m, 5H), 1.51-1.41 (m, 5H), 1.34 (m, 1H), 0.87 (d, J=6.60 Hz, 3H), 0.81 (d, J=6.65 Hz, 3H)

¹³C NMR (125 MHz, d⁶-dmso) δ=208.8, 151.1, 142.2, 114.4, 114.3, 99.5, 76.0, 66.7, 55.2, 52.3, 40.3, 33.9, 31.3, 24.8, 24.2, 22.6, 22.5, 22.4

### Example 2

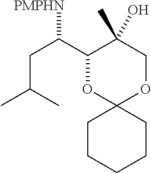

To the ketone (94 mg, 0.26 mmol) of Example 1 in Et₂O (10 mL) at −50° C. was added MeMgBr (0.156 mL, 3M in Et₂O) and the reaction was stirred for 1.5 h. Water was added and warmed to rt. The aqueous layer was extracted with Et₂O, the combined organic layer was dried and the solvent removed under reduced pressure. Purification by column chromatography (silicagel, toluene:EtOAc 4:1) gave 45 mg (46%) of the desired product >99% ee.

¹H NMR (500 MHz, d⁶-dmso) δ=6.76 (m, 4H), 5.58 (s, 1H(OH)), 4.63 (d, J=9.77 Hz, 1H), 3.69 (d, J=1.26 Hz, 1H), 3.66 (s, 3H), 3.65 (d, J=10.97 Hz, 1H), 3.60 (m, 1H), 3.44 (ddd, J=5.25, 6.82, 13.72 Hz, 1H), 3.26 (d, J=11.66 Hz, 1H), 2.08 (m, 1H), 1.72 (m, 1H), 1.55 (m, 6H), 1.44 (m, 2H), 1.32 (m, 2H), 1.25 (m, 2H), 1.04 (s, 3H), 0.86 (d, J=6.62 Hz, 3H), 0.81 (d, J=6.62 Hz, 3H)

¹³C NMR (125 MHz, d⁶-dmso) δ=151.9, 141.2, 116.0, 114.5, 98.0, 71.5, 68.7, 68.1, 56.0, 55.2, 52.1, 40.3, 37.3, 27.3, 25.2, 24.0, 23.2, 22.4, 22.3, 22.2, 21.8, 18.5

### Example 3

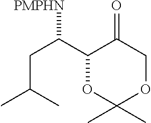

p-Anisidine (1.61 g, 13.1 mmol) was dissolved in dmso (30 mL) and isovaleraldehyde (1.03 g, 11.9 mmol) was added and stirred for 15 min. Then (L)-alanine (318 mg, 3.57 mmol), water (429 mg, 23.8 mmol) and ketoacetonide (5 g, 23.8 mmol) was added. After 24 h brine and water was added and the mixture was extracted with EtOAc. The combined organic layer was dried over Na₂SO₄ and the solvent removed under reduced pressure. Purification by column chromatography (Silicagel, DCM) was followed by crystallization from EtOH to give 2.7 g (31%) of the Mannich product (82% ee).

¹H NMR (500 MHz, d⁶-dmso) δ=6.68 (d, J=9.14 Hz, 2H), 6.62 (d, J=9.14 Hz, 2H), 4.50 (d, J=10.09 Hz, 1H), 4.36 (s, 1H), 4.19 (dd, J=0.63, 17.02 Hz, 1H), 3.92 (d, J=16.39 Hz, 1H), 3.81 (m, 1H), 3.62 (s, 3H), 1.62 (m, 1H), 1.45 (s, 3H), 1.44-1.35 (m, 2H), 1.42 (s, 3H), 0.87 (d, J=6.62 Hz, 3H), 0.80 (d, J=6.62 Hz, 3H)

### Example 4

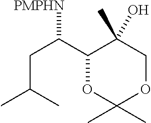

To the ketone (50 mg, 0.15 mmol) of Example 3 in Et₂O (2 mL) at −10° C. was added MeMgBr (0.056 mL, 3M in Et₂O) and the reaction was stirred for 1.5 h. Water was added and the aqueous layer extracted with Et₂O, the combined organic layer was dried and the solvent removed under reduced pressure. Purification by column chromatography (silicagel, toluene:EtOAc 4:1) gave 22 mg (42%) of the desired product with a dr of 5:1.

¹H NMR (500 MHz, C₆D₆) δ=6.70 (d, J=8.92 Hz, 2H), 6.47 (d, J=8.91 Hz, 2H), 3.75 (d, J=11.98 Hz, 1H), 3.74 (m, 1H), 3.59 (s, 1H), 3.50 (d, J=11.66 Hz, 1H), 3.33 (s, 3H), 1.57 (s, 3H), 1.55 (m, 1H), 1.47 (m, 1H), 1.35 (m, 1H), 1.25 (s, 3H), 1.08 (s, 3H), 0.77 (d, J=6.30 Hz, 6H)

¹³C NMR (125 MHz, C₆D₆) δ=154.4, 139.9, 117.9, 115.1, 99.2, 73.0, 70.4, 68.8, 55.1, 53.1, 40.2, 28.6, 25.1 23.7, 23.4, 21.6, 19.6

### Example 5

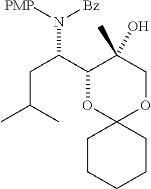

To MgBr₂Et₂O (134 mg, 0.52 mmol) in DCM (2.6 mL) was added Bz₂O (124 mg, 0.52 mmol), NEt₃ (110 μL, 0.78 mmol) and the amine (100 mg, 0.26 mmol) of Example 2. The mixture was warmed to 35° C. for 18 h. The reaction was quenched by the addition of water and the aqueous layer was extracted with DCM. The combined organic layer was dried and the solvent removed under reduced pressure. Purification by column chromatography (silicagel, toluene:EtOAc 4:1) gave the desired product.

¹H NMR (500 MHz, d⁶-dmso) δ=7.57 (m, 2H), 7.34 (m, 2H), 7.24-7.17 (m, 4H), 6.83 (m, 2H), 4.63 (d, J=8.51 Hz, 1H), 4.37 (d, J=12.61 Hz, 1H), 4.14 (ddd, J=2.36, 9.30, 9.14 Hz, 1H), 3.96 (d, J=12.61 Hz, 1H), 3.70 (s, 3H), 2.79 (ddd, J=2.31, 11.98, 13.35 Hz, 1H), 2.00 (m, 1H), 1.85 (m, 2H), 1.64 (m, 4H), 1.58 (s, 3H), 1.52-1.29 (m, 6H), 0.93 (d, J=6.62 Hz, 3H), 0.74 (d, J=6.30 Hz, 3H)

### Example 6

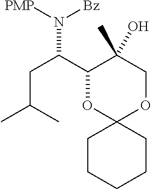

To the amine (2 g, 5.3 mmol) of Example 2 and K₂CO₃ (1.24 g, 9.01 mmol) in acetonitrile (20 mL) was added benzyl bromide (1.54 g, 9.01 mmol) and the mixture was stirred at 70° C. for 3 h. Water was added and the organic solvent removed under reduced pressure. The aqueous layer was extracted with EtOAc and the combined organic layer was washed with water, dried over Na₂SO₄ and the solvent was evaporated. Purification by column chromatography (silicagel, toluene:EtOAc 10:1) gave 1.6 g (65%) of the desired product.

¹H NMR (500 MHz, d⁶-dmso) δ=7.28 (m, 2H), 7.21 (m, 2H), 7.10 (m, 1H), 6.91 (d, J=9.45 Hz, 2H), 6.69 (d, J=9.15 Hz, 2H), 5.44 (s, 1 OH), 4.65 (d, J=16.10 Hz, 1H), 4.36 (d, J=16.05 Hz, 1H), 3.92 (dd, J=6.45, 12.45 Hz, 1H), 3.77 (d, J=5.05 Hz, 1H), 3.61 (s, 3H), 3.59 (d, J=11.95 Hz, 1H), 3.34 (d, J=11.65 Hz, 1H), 1.89 (m, 1H), 1.63 (m, 2H), 1.58 (m, 2H), 1.46 (m, 2H), 1.41-1.25 (m, 6H), 1.10 (s, 3H), 0.84 (d, J=6.00 Hz, 3H), 0.73 (d, J=6.30 Hz, 3H)

¹³C NMR (125 MHz, d⁶-dmso) δ=152.3, 140.0, 127.9, 127.5, 126.1, 125.3, 119.2, 113.7, 98.1, 74.4, 68.8, 68.4, 60.2, 55.0, 38.1, 36.9, 27.5, 25.2, 24.6, 23.3, 22.7, 22.4, 22.2, 221, 21.0

### Example 7

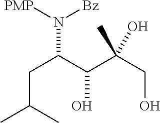

The acetal (900 mg, 1.92 mmol) of Example 6 was dissolved in a mixture of 37% aqueous HCl (9.5 mL), water (9.5 mL) and DMF (5 mL). After 30 min at 50° C. the reaction was cooled to it, EtOAc (30 mL) added and the pH adjusted to pH 7. The aqueous layer was extracted with EtOAc and the combined organic layer was washed with water, dried over Na₂SO₄ and the solvent was removed under reduced pressure to give 963 mg (still wet, quant. yield).

¹H NMR (500 MHz, d⁶-dmso) δ=7.23-7.17 (m, 4H), 7.09 (m, 1H), 6.77 (d, J=9.10 Hz, 2H), 6.67 (d, J=9.15 Hz, 2H), 4.63 (d, J=16.70 Hz, 1H), 4.51 (t, J=5.67 Hz, 1 OH), 4.49 (d, J=17.05 Hz, 1H), 4.48 (d, J=6.00 Hz, 1 OH), 4.02 (q, J=5.88 Hz, 1H), 3.60 (s, 3H), 3.49 (m, 1H), 3.48 (m, 1H), 3.37 (dd, J=10.56, 5.83 Hz, 1H), 1.59 (m, 1H), 1.57 (m, 2H), 1.11 (s, 3H), 0.80 (d, J=5.99 Hz, 3H), 0.77 (d, J=5.99 Hz, 3H) ¹³C NMR (125 MHz, d⁶-dmso) δ=151.3, 144.0, 140.3, 127.9, 127.0, 125.8, 117.4, 113.9, 76.1, 74.3, 66.7, 59.1, 55.0, 47.7, 40.3, 24.8, 22.9, 22.8, 22.4

### Example 8

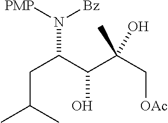

To the triol (120 mg, 0.309 mmol) of Example 7 in Et₂O (3 mL) at rt was added triethylamine (64 μL, 0.464 mmol). After 10 min the reaction mixture was cooled to 0° C. and acetylchloride (29 μL, 0.402 mmol) was added. After 1 h sat. NH₄Cl solution was added. The aqueous layer was extracted with Et₂O and the combined organic layer was washed with water, dried over Na₂SO₄ and the solvent was removed under reduced pressure. Purification is possible by column chromatography and gave 126 mg (95%) of the acetate.

¹H NMR (500 MHz, d⁶-dmso) δ=7.23 (m, 2H), 7.18 (m, 2H), 7.09 (m, 1H), 6.80 (d, J=9.15 Hz, 2H), 6.67 (d, J=9.15 Hz, 2H), 4.99 (d, J=4.75 Hz, 1 OH), 4.92 (s, 1 OH), 4.75 (d, J=16.70 Hz, 1H), 4.41 (d, J=16.70 Hz, 1H), 4.16 (m, 2H), 4.07 (m, 1H), 3.60 (s, 3H), 3.43 (dd, J=6.13, 3.62 Hz, 1H), 2.02 (s, 3H), 1.63 (m, 1H), 1.55 (m, 2H), 1.18 (s, 3H), 0.77 (d, J=5.99 Hz, 6H)

¹³C NMR (125 MHz, d⁶-dmso) δ=170.4, 151.4, 144.0, 140.1, 127.9, 127.0, 125.8, 117.7, 113.9, 77.4, 73.2, 68.6, 64.9, 58.9, 54.9, 49.3, 40.3, 24.7, 23.2, 22.8, 22.4, 20.8

### Example 9

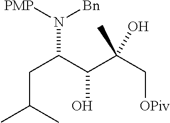

To the triol (21 mg, 0.054 mmol) of Example 7 in Et₂O (3 mL) at it was added triethylamine (12 μL, 0.081 mmol). After 10 min pivaloylchloride (7 μL, 0.06 mmol) was added. After 1 h and 3 h an additional amount of triethylamine and pivaloylchloride was added. The reaction was stirred at it over night, sat. NH₄Cl solution was added. The aqueous layer was extracted with Et₂O and the combined organic layer was washed with water, dried over Na₂SO₄ and the solvent was removed under reduced pressure. Purification from starting material is possible by column chromatography.

¹H NMR (500 MHz, d⁶-dmso) δ=7.23 (m, 2H), 7.18 (m, 2H), 7.10 (m, 1H), 6.80 (d, J=9.14 Hz, 2H), 6.67 (d, J=9.14 Hz, 2H), 4.90 (d, J=6.30 Hz, 1OH), 4.88 (s, 1OH), 4.74 (d, J=16.70 Hz, 1H), 4.43 (d, J=16.39 Hz, 1H), 4.17 (d, J=11.03 Hz, 1H), 4.08 (d, J=10.71 Hz, 1H), 4.06 (m, 1H), 3.60 (s, 3H), 3.44 (dd, J=6.15, 3.94 Hz, 1H), 1.64 (m, 1H), 1.54 (m, 2H), 1.19 (s, 3H), 1.16 (s, 9H), 0.78 (d, J=6.31 Hz, 3H), 0.77 (d, J=6.30 Hz, 3H)

¹³C NMR (125 MHz, d⁶-dmso) δ=177.3, 151.4, 140.1, 128.9, 128.2, 127.9, 127.0, 125.8, 117.7, 113.9, 77.1, 73.4, 68.7, 58.9, 54.9, 40.3, 36.5, 26.8, 26.0, 24.7, 23.0, 22.9, 22.4

### Example 10

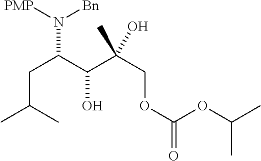

To the triol (50 mg, 0.129 mmol) of Example 7 in toluene (0.5 mL) at it was added triethylamine (30 mg, 0.297 mmol) followed by isopropylchloroformate (0.270 mL, 1M in tol). The mixture was stirred at it overnight. An aqueous sat. NH₄Cl solution was added and the aqueous layer extracted, combined and dried over Na₂SO₄ and the solvent was removed under reduced pressure. Purification by column chromatography gave 60 mg (98%) of the isopropyl carbonate.

¹H NMR (500 MHz, d⁶-dmso) δ=7.23 (d, J=7.25 Hz, 2H), 7.18 (t, J=7.56 Hz, 2H), 7.09 (t, J=7.25 Hz, 1H), 6.81 (d, J=9.46 Hz, 2H), 6.67 (d, J=9.14 Hz, 2H), 5.01 (s, 1 OH), 4.97 (d, J=6.31 Hz, 1 OH), 4.76 (d, J=16.49 Hz, 1H), 4.75 (q, J=6.09 Hz, 1H), 4.40 (d, J=16.71 Hz, 1H), 4.25 (d, J=10.72 Hz, 1H), 4.18 (d, J=10.72 Hz, 1H), 4.08 (m, 1H), 3.61 (s, 3H), 3.41 (dd, J=3.78, 5.99 Hz, 1H), 1.63 (m, 1H), 1.54 (m, 2H), 1.22 (d, J=6.30 Hz, 1H), 1.21 (d, J=5.61 Hz, 3H), 1.19 (s, 3H), 0.76 (d, J=5.80 Hz, 6H)

¹³C NMR (125 MHz, d⁶-dmso) δ=154.3, 151.5, 144.0, 140.1, 127.8, 127.0, 125.7, 117.7, 113.8, 77.3, 73.1, 71.9, 71.1, 58.9, 54.9, 40.2, 24.7, 23.1, 22.8, 22.3, 21.5

### Example 11

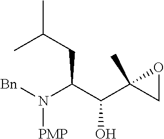

Via Mesylation (in situ)

34.5 mg (89 μmol) of the unprotected triol of Example 7 was dissolved in 5 ml dichloromethane and charged with 15 mg mesylchloride (1.5 eq). To the solution 1 ml pyridine was added at room temperature and the mixture was stirred until complete conversion was observed (HPLC). To the reaction mixture 5 ml of dichloromethane and 10 ml of saturated ammonium chloride was added. The organic phase was separated and washed with saturated ammonium chloride again while pH was adjusted to 3 via adding 2 M HCl. The organic phase was reduced to dryness and 30 mg (91%) of a glass like solid was isolated.

¹H NMR (300 MHz, CDCl₃) δ=7.72 (d, 2H), 7.27 (m, 3H), 6.93 (d, 2H), 6.85 (d, 2H), 5.07 (d, 2H), 4.94 (d, 2H), 4.48 (m, 1H), 3.79 (s, 3H), 3.67 (m, 1H), 2.94 (d, 1H), 2.71 (d, 1H), 1.78 (m, 1H), 1.62 (s, 3H), 1.35 (m, 2H), 0.92 (d, 3H), 0.68 (d, 3H).

From Example 8:

30.3 mg of the acetate of Example 8 was dissolved in 5 ml dichloromethane. To the solution 240 mg potassium tert-butyrat was added. The mixture was stirred for 22 h until complete conversion was observed. After hydrolysis the organic phase was separated and reduced to dryness gaining 30 mg (quant.) of the product.

From Example 9:

70 mg of carbonate of Example 9 were dissolved in 5 ml dichloromethane. To the solution 1 ml pyridine was added and the mixture was stirred at room temperature for 51 h and afterwards for 2.5 h under reflux. 86% conversion to the desired product was observed.

1. A method for producing Carfilzomib according to Formula 13, 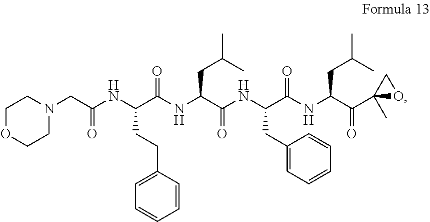 [SMILES: CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)[C@@]1(C)CO1]
comprising the steps:
a) organocatalytic Mannich-reaction of compounds of Formula 1, 2 and 3, 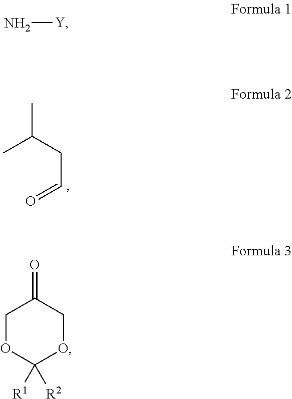
wherein
Y stands for an aromate, heteroaromate or a substituted aromate/heteroaromate,
R¹, R² stand for an alkyl, wherein R¹ and R² can be connected, forming a ring of 4, 5, 6, or 7 atoms,
leading to a compound of Formula 4, 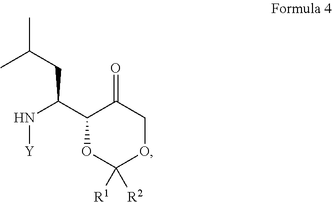
b) methyl addition to the compound of Formula 4, optionally followed by protection of the nitrogen, leading to a compound of Formula 5, 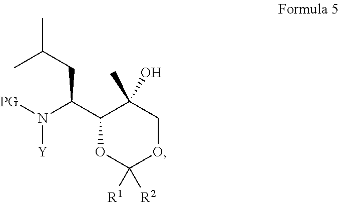
wherein PG stands for a protecting group,
and
c) deprotection of the compound of Formula 5 to a compound of Formula 6, 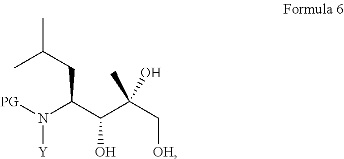
and converting the compound of Formula 6 into Carfilzomib.

2. The method according to claim 1, further comprising the steps of
d1) transforming the primary alcohol in the compound of Formula 6 into a leaving group,
e1) deprotecting the amino-function to obtain a compound of Formula 7, 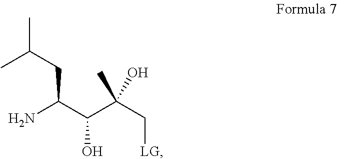
wherein
LG stands for a leaving group
f1) coupling the compound of Formula 7 to the peptide of Formula 8, 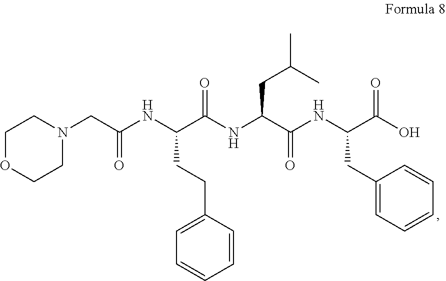 [SMILES: CC(C)C[C@H](NC(=O)[C@H](CCc1ccccc1)NC(=O)CN1CCOCC1)C(=O)N[C@@H](Cc1ccccc1)C(=O)O]
leading to a peptide of Formula 9, 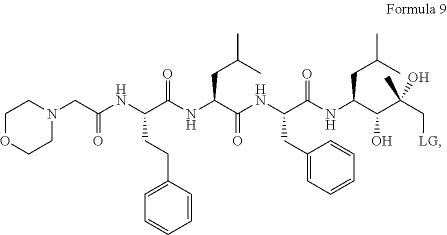
and converting the compound of Formula 9 into Carfilzomib.

3. The method according to claim 2, wherein the compound of Formula 9 is converted into Carfilzomib by the steps of
g1.1) epoxide formation by base addition, and
h1.1) oxidation of the secondary alcohol.

4. The method according to claim 2, further comprising the steps of
g1.2) oxidation of the secondary alcohol in the compound of Formula 9, and
h1.2) epoxide formation by base addition.

5. The method according to claim 1, further comprising the steps of
d2) transforming the primary alcohol in the compound of Formula 6 into a leaving group leading to a compound of Formula 10, 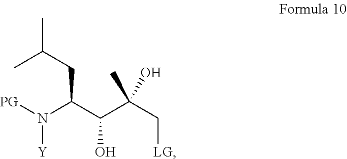
wherein
LG stands for a leaving group
PG stands for a protecting group
Y stands for an aromate, heteroaromate or a substituted aromate/heteroaromate,
e2) oxidizing the secondary alcohol in the compound of Formula 10 to obtain a compound a Formula 11, 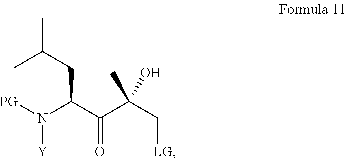
f2) epoxide formation by base addition,
g2) deprotection of the amine leading to an epoxide of Formula 12, 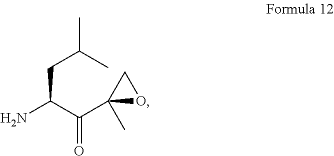
and
h2) coupling of the epoxide of Formula 12 to a peptide of Formula 8,

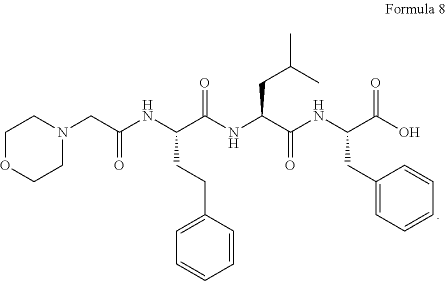 [SMILES: CC(C)C[C@H](NC(=O)[C@H](CCc1ccccc1)NC(=O)CN1CCOCC1)C(=O)N[C@@H](Cc1ccccc1)C(=O)O]

6. A method for producing an epoxide of Formula 12 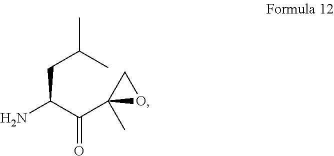
comprising the steps
a) organocatalytic Mannich-reaction of compounds of Formula 1, 2 and 3, 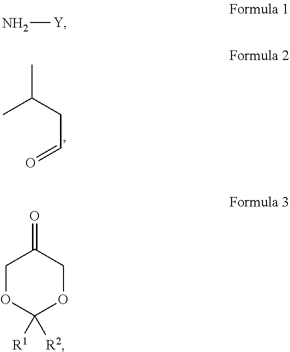
wherein
Y stands for an aromate, heteroaromate or a substituted aromate/heteroaromate,
R¹, R² stand for an alkyl, wherein R¹ and R² can be connected, forming a ring of 4, 5, 6, or 7 atoms,
leading to a compound of Formula 4, 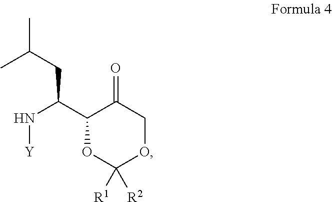
b) methyl addition to the compound of Formula 4, optionally followed by protection of the nitrogen, leading to a compound of Formula 5, 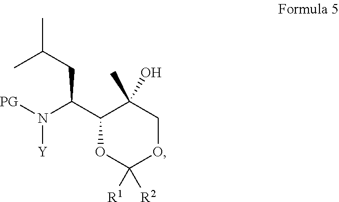
wherein PG stands for a protecting group,
and
c) deprotection of the compound of Formula 5 to a compound of Formula 6, 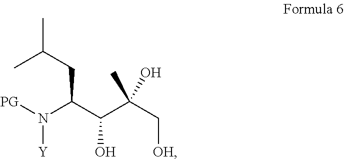
d) transforming the primary alcohol in the compound of Formula 6 into a leaving group leading to a compound of Formula 10, 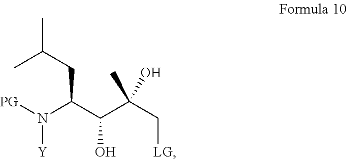
wherein
LG stands for a leaving group
e) oxidizing the secondary alcohol in the compound of Formula 10 to obtain a compound of Formula 11, 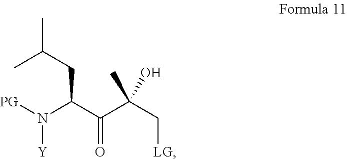
f) epoxide formation by base addition,
g) deprotection of the amine.

7. The method according to claim 6, wherein the base is pyridine, triethylamine or sodium/potassium tert-butanolate.

8. The method according to claim 6, wherein the compound of Formula 3 is 2,2-Dimethyl-1,3-dioxan-5-one or 1,5-Dioxaspiro[5.5]undecan-3-one.

9. The method according to claim 6, wherein step a) is carried out with (L)-Alanine as catalyst.

10. The method according to claim 6, wherein step b) is carried out with a Grignard reagent.

11. A compound selected from the group consisting of:

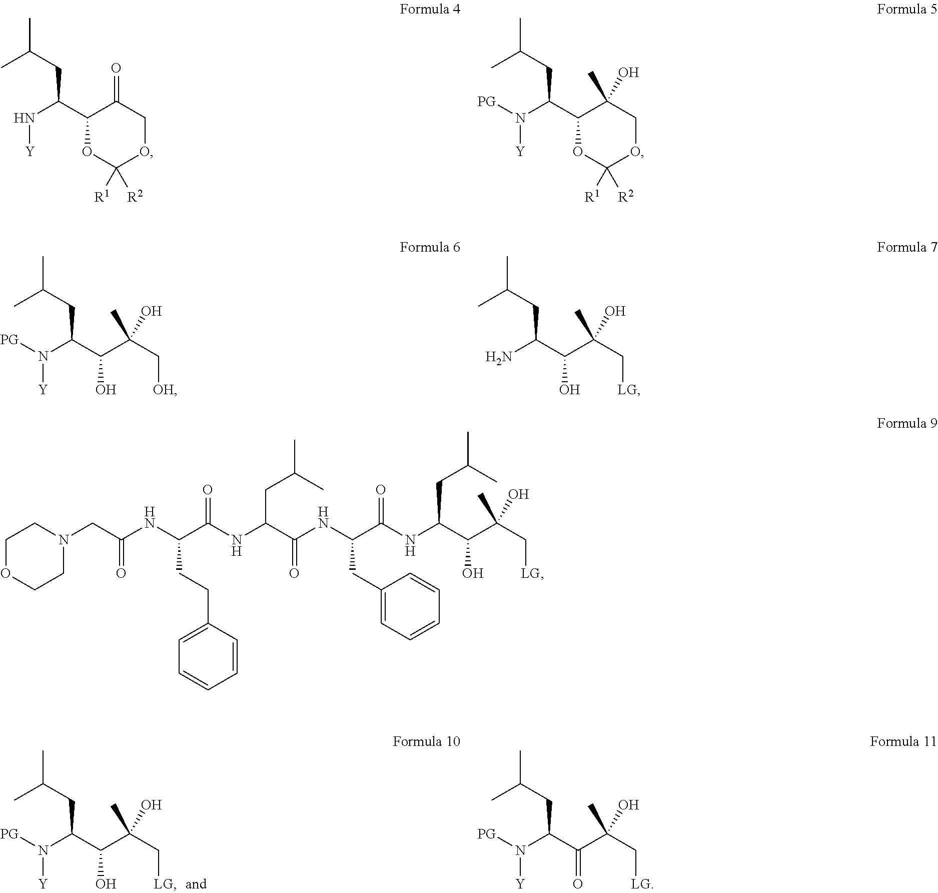

12.-17. (canceled)

18. The method according to claim 3, wherein the base is pyridine, triethylamine or sodium/potassium tert-butanolate.

19. The method according to claim 1, wherein the compound of Formula 3 is 2,2-Dimethyl-1,3-dioxan-5-one or 1,5-Dioxaspiro[5.5]undecan-3-one.

20. The method according to claim 1, wherein step a) is carried out with (L)-Alanine as catalyst.

21. The method according to claim 1, wherein step b) is carried out with a Grignard reagent.
